(19) United States
(12) Patent Application Publication
(10) Pub. No.: US 2019/0000034 A1
(43) Pub. Date: Jan. 3, 2019

(54) Animal Kennel System

(71) Applicant: Denise Allen, Pinellas Park, FL (US)

(72) Inventor: Denise Allen, Pinellas Park, FL (US)

(21) Appl. No.: 15/638,531

(22) Filed: Jun. 30, 2017

(51) Int. Cl. A01K 1/03; A01K 1/02; A01K 1/00

(52) U.S. Cl. CPC A01K 1/034; A61M 2021/0044; A01K 1/0035; A01K 1/0245

(57) ABSTRACT

An animal kennel system for soothing an animal in a kennel includes a kennel that may contain an animal. An enclosure is removably positioned on the kennel to completely surround the kennel. The enclosure is comprised of an opaque material to reduce light entering the kennel thereby soothing the animal in the kennel. A pocket is coupled to the enclosure and the pocket receives a picture. The pocket is comprised of a translucent material to facilitate the picture to be visible.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to kennel devices and more particularly pertains to a new kennel device for soothing an animal in a kennel.

### BRIEF SUMMARY OF THE INVENTION

An embodiment of the disclosure meets the needs presented above by generally comprising a kennel that may contain an animal. An enclosure is removably positioned on the kennel to completely surround the kennel. The enclosure is comprised of an opaque material to reduce light entering the kennel thereby soothing the animal in the kennel. A pocket is coupled to the enclosure and the pocket receives a picture. The pocket is comprised of a translucent material to facilitate the picture to be visible.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an animal kennel system according to an embodiment of the disclosure.

FIG. 2 is a front perspective view of an embodiment of the disclosure showing a first and second curtain being closed.

FIG. 3 is a top perspective view of an embodiment of the disclosure.

FIG. 4 is a front exploded view of an embodiment of the disclosure.

FIG. 5 is a bottom view of an embodiment of the disclosure.

### DETAILED DESCRIPTION OF THE INVENTION

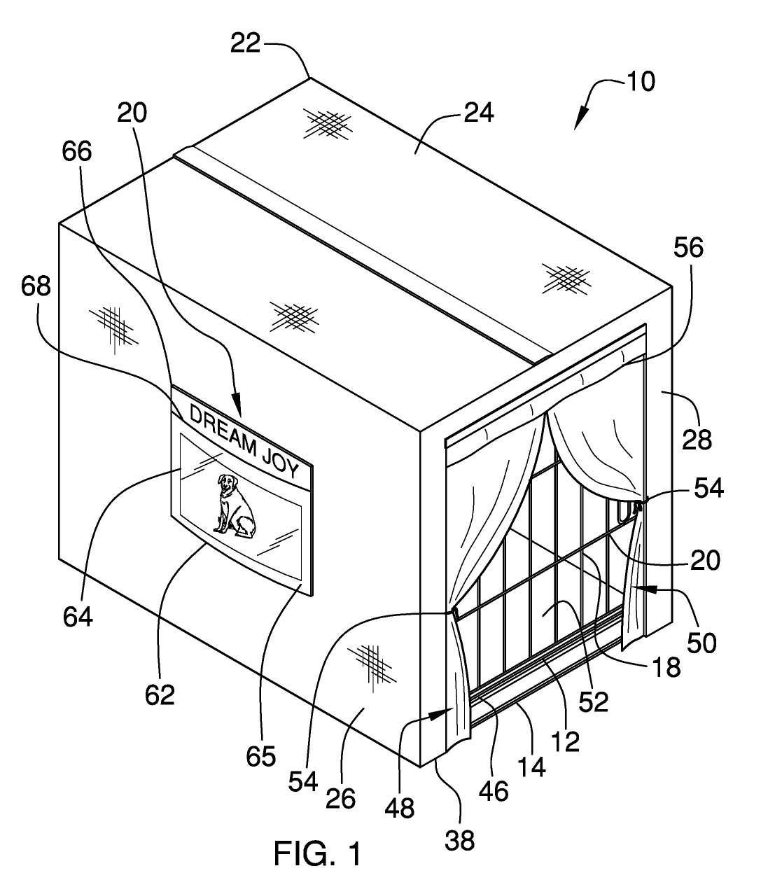

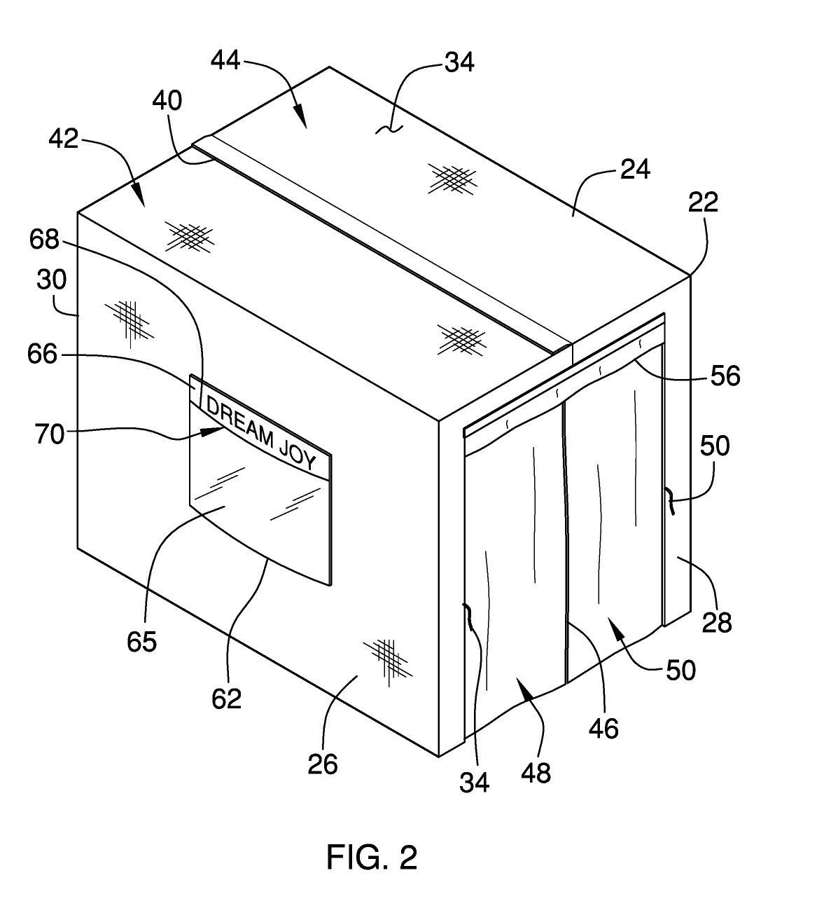

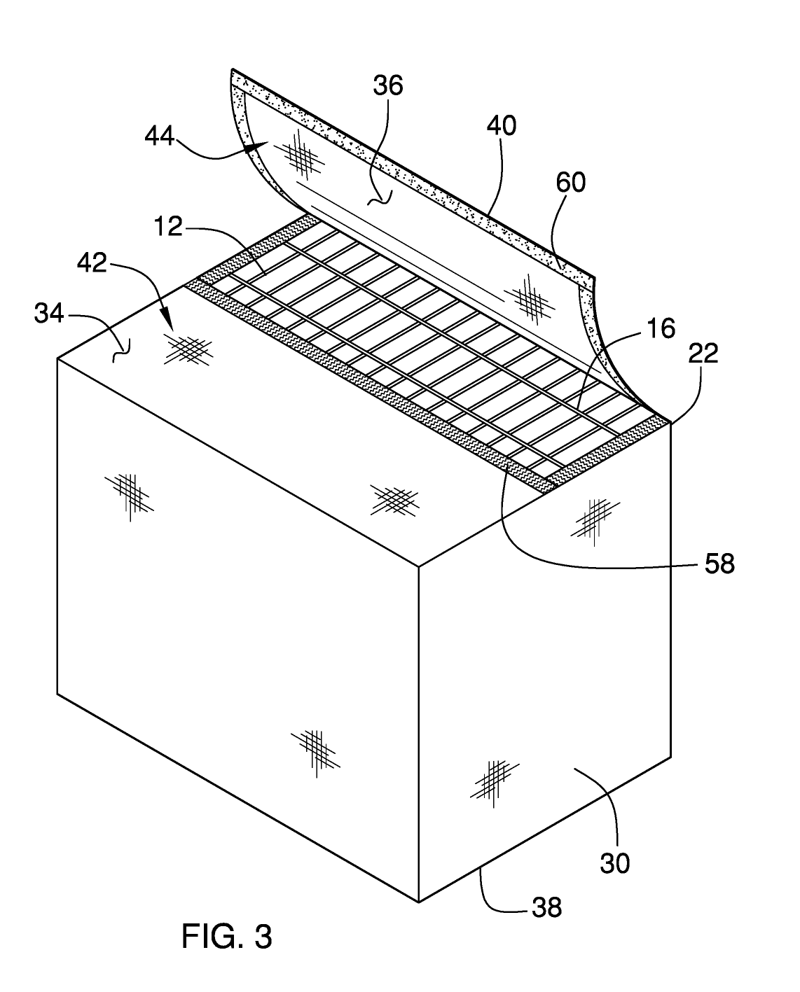

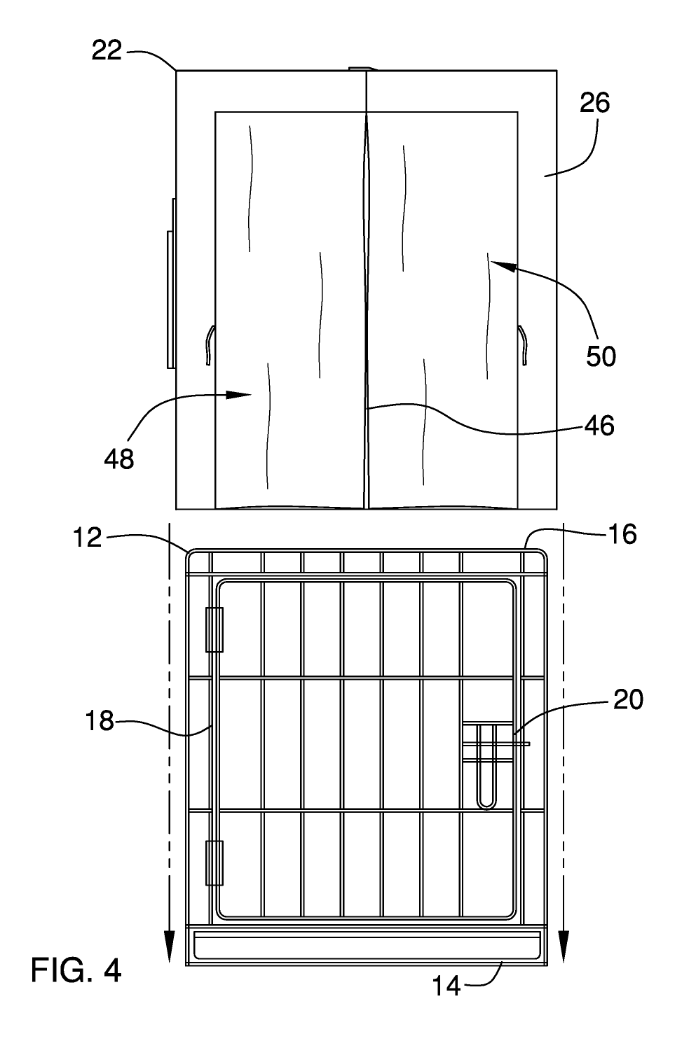

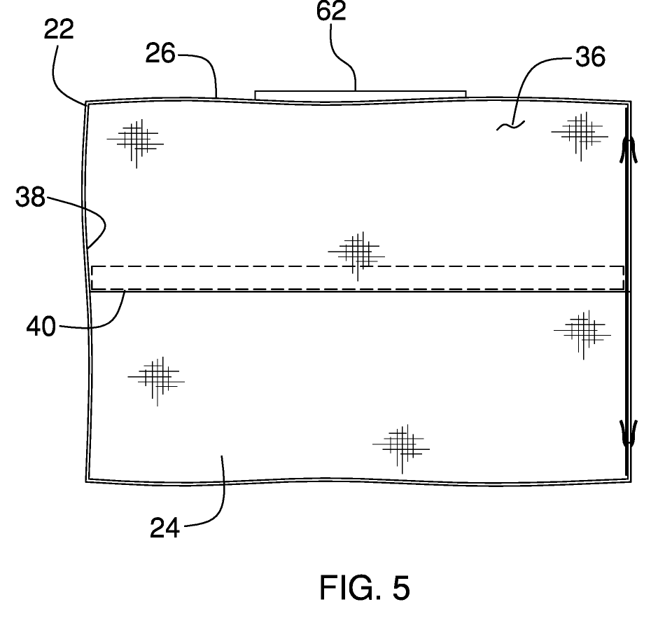

With reference now to the drawings, and in particular to FIGS. 1 through 5 thereof, a new kennel device embodying the principles and concepts of an embodiment of the disclosure and generally designated by the reference numeral 10 will be described.

As best illustrated in FIGS. 1 through 5, the animal kennel system 10 generally comprises a kennel 12 that may contain an animal. The animal may be a dog, a cat or any other domesticated animal. The kennel 12 has a bottom wall 14, a top wall 16 and an outer wall 18 extending therebetween. The outer wall 18 has a front side 20 and the front side 20 is selectively opened to access an interior of the kennel 12. Each of the top wall 16 and the outer wall 18 comprises a cage. Additionally, the kennel 12 may be a collapsible kennel 12 or the like.

An enclosure 22 is provided and the enclosure 22 is removably positioned on the kennel 12 having the enclosure 22 completely surrounding the kennel 12. The enclosure 22 is comprised of a deformable material such as textile or the like. Additionally, the enclosure 22 is comprised of an opaque material to reduce light entering the kennel 12 thereby soothing the animal in the kennel 12. The enclosure 22 has an upper wall 24 and a perimeter wall 26 extending downwardly therefrom. The perimeter wall 26 has a forward side 28, a back side 30, a first lateral side 32, a first surface 34, a second surface 36 and a distal edge 38 with respect to the upper wall 24. The upper wall 24 lies on the top wall 16 of the kennel 12 and the perimeter wall 26 extends downwardly along the outer wall 18 when the enclosure 22 is positioned on the kennel 12.

The upper wall 24 has a first cut 40 extending between the front side 20 and the back side 30 to define a first flap 42 and a second flap 44. The first flap 42 is selectively matable to the second flap 44 to retain the enclosure 22 on the kennel 12. Additionally, the forward side 28 has a second cut 46 extending upwardly from the distal edge 38 of the forward side 28 toward the upper wall 24 to define a first curtain 48 and a second curtain 50. Each of the first 48 and second 50 curtains is selectively drawn apart from each other to expose the front side 20 of the kennel 12. A cushion 52 is provided and the cushion 52 is selectively placed upon the bottom wall 14 of the kennel 12. Thus, the animal may lie on the cushion 52 when the animal is in the kennel. The cushion 52 is comprised of a resiliently compressible material to enhance comfort for the animal.

A pair of ties 54 is provided and each of the ties 54 is coupled to the forward side of the enclosure 22. Each of the ties 54 is aligned with an associated one of the first 48 and second 50 curtains. Moreover, each of the ties 54 is selectively tied around the associated first 48 and second 50 curtain to retain each of the first 48 and second 50 curtains being drawn apart from each other. A valence 56 is coupled to the forward side 28 of the enclosure 22. The valence 56 is horizontally oriented and is aligned with the first and second curtain 50s. In this way the first 48 and second 50 curtains and the valence 56 simulate drapery on a window or the like.

A first mating member 58 is coupled to the first surface 34 of the first flap 42 and the first mating member 58 is aligned with the first cut 40. A second mating member 60 is coupled to the second surface 36 of the second flap 44 and the second mating member 60 is aligned with the first cut 40. The second mating member 60 releasably engages the first mating member 58 to close the first 42 and second 44 flaps. Each of the first 58 and second 60 mating members may comprise a hook and loop fastener or the like. Additionally, each of the first 58 and second 60 mating members may extend around an entire perimeter of the associated first 42 and second 44 flaps.

A pocket 62 is coupled to the enclosure 22 and the pocket 62 may receive a picture 64. The picture 64 may be a photo of the animal in the kennel 12 or any other selected image. The pocket 62 is comprised of a translucent material thereby facilitating the picture 64 to be visible. The pocket 62 has a first panel 65 that is spaced from a second panel 66 and the first panel 65 has a top edge 68. The top edge 68 is open to receive the picture 64 having the picture 64 being positioned between the first 64 and second 66 panels. The second panel 66 is coupled to the first lateral side 32 of the enclosure 22 and indicia 70 are printed on the second panel 66. The indicia 70 comprise words and the words may be “Dream Joy”.

In use, the enclosure 22 is positioned on the kennel 12 and the first mating member 58 is mated to the second mating member 60. In this way the enclosure 22 is retained on the kennel 12 to soothe the animal in the kennel 12 when the animal is comforted by an enclosed space. The first 48 and second 50 curtains are selectively drawn apart to expose the front side 20 of the kennel 12. Each of the ties 54 is selectively tied around the corresponding first 48 and second 50 curtain during day time and the like. Each of the first 48 and second 50 curtains is selectively suspended on the front side 20 of the kennel 12 during night time and the like. Additionally, the picture 64 is positioned in the pocket 62 such that the picture 64 is visible to a user.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An animal kennel system comprising:
a kennel being configured to contain an animal;
an enclosure being removably positioned on said kennel having said enclosure completely surrounding said kennel, said enclosure being comprised of an opaque material wherein said enclosure is configured to reduce light entering said kennel thereby soothing the animal in said kennel; and
a pocket being coupled to said enclosure wherein said pocket is configured to receive a picture, said pocket being comprised of a translucent material wherein said pocket is configured to facilitate the picture to be visible.

2. The system according to claim 1, wherein said kennel has a bottom wall, a top wall and an outer wall extending therebetween, said outer wall having a front side, said front side being selectively opened to access an interior of said kennel, each of said top wall and said outer wall comprising a cage.

3. The system according to claim 2, wherein said enclosure has an upper wall and a perimeter wall extending downwardly therefrom, said perimeter wall having a forward side, a back side, a first lateral side, a first surface, a second surface and a distal edge with respect to said upper wall, said upper wall lying on said top wall of said kennel having said perimeter wall extending downwardly along said outer wall when said enclosure is positioned on said kennel.

4. The system according to claim 3, wherein said upper wall has a first cut extending between said front side and said back side to define a first flap and a second flap, said first flap being selectively matable to said second flap to retain said enclosure on said kennel.

5. The system according to claim 3, wherein said forward side has a second cut extending upwardly from said distal edge of said forward side toward said upper wall to define a first curtain and a second curtain, each of said first and second curtains being selectively drawn apart from each other to expose said front side of said kennel.

6. The system according to claim 5, further comprising a pair of ties, each of said ties being coupled to said forward side of said enclosure, each of said ties being aligned with an associated one of said first and second curtains, each of said ties being selectively tied around said associated first and second curtain to retain each of said first and second curtains being drawn apart from each other.

7. The system according to claim 6, further comprising a valence being coupled to said forward side of said enclosure, said valence being horizontally oriented and being aligned with said first and second curtains.

8. The system according to claim 4, further comprising a first mating member being coupled to said first surface of said first flap, said first mating member being aligned with said first cut.

9. The system according to claim 8, further comprising a second mating member being coupled to said second surface of said second flap, said second mating member being aligned with said first cut, said second mating member releasably engaging said first mating member to close said first and second flaps.

10. The system according to claim 3, wherein said pocket has a first panel being spaced from a second panel, said first panel having a top edge, said top edge being open wherein said top edge is configured to receive the picture having the picture being positioned between said first and second panels, said second panel being coupled to said first lateral side of said enclosure, said second panel having indicia being printed thereon, said indicia comprising words.

11. An animal kennel system comprising:
a kennel being configured to contain an animal, said kennel having a bottom wall, a top wall and an outer wall extending therebetween, said outer wall having a front side, said front side being selectively opened to access an interior of said kennel, each of said top wall and said outer wall comprising a cage; and
an enclosure being removably positioned on said kennel having said enclosure completely surrounding said kennel, said enclosure being comprised of an opaque material wherein said enclosure is configured to reduce light entering said kennel thereby soothing the animal in said kennel, said enclosure having an upper wall and a perimeter wall extending downwardly therefrom, said perimeter wall having a forward side, a back side, a first lateral side, a first surface, a second surface and a distal edge with respect to said upper wall, said upper wall lying on said top wall of said kennel having said perimeter wall extending downwardly along said outer wall when said enclosure is positioned on said kennel, said upper wall having a first cut extending between said front side and said back side to define a first flap and a second flap, said first flap being selectively matable to said second flap to retain said enclosure on said kennel, said forward side having a second cut extending upwardly from said distal edge of said forward side toward said upper wall to define a first curtain and a second curtain, each of said first and second curtains being selectively drawn apart from each other to expose said front side of said kennel;
a pair of ties, each of said ties being coupled to said forward side of said enclosure, each of said ties being aligned with an associated one of said first and second curtains, each of said ties being selectively tied around said associated first and second curtain to retain each of said first and second curtains being drawn apart from each other;
a valence being coupled to said forward side of said enclosure, said valence being horizontally oriented and being aligned with said first and second curtains;
a first mating member being coupled to said first surface of said first flap, said first mating member being aligned with said first cut;
a second mating member being coupled to said second surface of said second flap, said second mating member being aligned with said first cut, said second mating member releasably engaging said first mating member to close said first and second flaps; and
a pocket being coupled to said enclosure wherein said pocket is configured to receive a picture, said pocket being comprised of a translucent material wherein said pocket is configured to facilitate the picture to be visible, said pocket having a first panel being spaced from a second panel, said first panel having a top edge, said top edge being open wherein said top edge is configured to receive the picture having the picture being positioned between said first and second panels, said second panel being coupled to said first lateral side of said enclosure, said second panel having indicia being printed thereon, said indicia comprising words.
